(19) United States
(12) Patent Application Publication
(10) Pub. No.: US 2004/0131643 A1
(43) Pub. Date: Jul. 8, 2004

(54) Immune response potentiation

(76) Inventors: Harleen Grewal, Bergen (NO); Halvor Sommerfelt, Bergen (NO)

Correspondence Address: SUGHRUE MION, PLLC, 2100 PENNSYLVANIA AVENUE, N.W., SUITE 800, WASHINGTON, DC, 20037, US

(21) Appl. No.: 10/469,920

(22) PCT Filed: Mar. 1, 2002
(86) PCT No.: PCT/GB02/00885

(30) Foreign Application Priority Data
Mar. 5, 2001 (NO) 20011108
Nov. 15, 2001 (NO) 20015591

(51) Int. Cl. A61K 39/106; A61K 33/32

(52) U.S. Cl. 424/261.1; 424/643

(57) ABSTRACT

The invention provides the use of a physiologically tolerable zinc compound for the manufacture of a medicament for use in a method of treatment of an animal to generate a mucosal immune response therein to an antigen, which method comprises administering to said animal a said physiologically tolerable zinc compound and a said antigen whereby to generate said response, preferably by bringing said zinc compound and said antigen into contact with a mucosal surface in said animal.

The present invention relates to a method for modulating mucosal immune responses to mucosally delivered antigens, and to pharmaceutical compositions and to kits for use therein.

Mucosal immune responses are initiated by transepithelial import of antigens from mucosal surfaces into organised lymphoid tissues located in the mucosa or in nearby lymph nodes where antigen-specific B-cells are activated. There is a complex cellular traffic linking this uptake of antigens at the sampling sites with the production of antibodies at widespread epithelial effector sites. Stimulation of the gut mucosal immune system is most efficiently achieved by antigens applied directly to the luminal surface of the intestine. It is recognised that, in order to efficacious, vaccines against enteric infections should induce a specific secretory IgA immune response in the gut. However, the mucosal immune response to many soluble antigens administered by the oral route is low and often requires large and frequently administered doses of antigen (see Holmgren et al., Vaccine 11:1179-1184 (1993)). In order to exploit the practicality of mucosal immunisation and the ability of this route to induce immune responses at mucosal surfaces, there is a need to develop substances that can enhance such responses. Moreover, as several diseases, including food allergies, coeliac disease and rheumatoid disease and other autoimmune disorders may be caused by or associated with harmfully exaggerated responses to otherwise innocuous substances or organisms, there is a need to identify substances that can mitigate such harmful responses.

It is thus an object of the present invention to provide a method and composition that provides an immunomodulating effect of the immune response to antigens administered either passively (as in food substances) or actively (as in vaccines delivered by the mucosal route, e.g. oral, nasal, pulmonary, rectal, etc).

Zinc affects many aspects of the immune system, and the gastrointestinal tract plays a central role in zinc homeostasis. Though much is still unclear about the specific physiological roles of zinc, the critical regulatory influence on the immune system is well established. The profound effect of zinc status on immune function in humans involves a complex interaction with crucial components of both the intra- and intercellular signalling systems of most immunocompetent cells, subsets of T-lymphocytes and monocytes being most affected in zinc deficiency states.

Humans appear to have the capacity to regulate whole body zinc content over a 10-fold change in intake. Short-term intake of zinc, even several-fold above the RDA, is generally considered safe in pre-school children and adults. Concomitant parenteral immunisation and moderately high-dose zinc supplementation has been undertaken with inconclusive results (see Provinciali et al. Age and Ageing 27:715-722 (1998), Rawer et al. Kidney Int. Suppl. 22:149-152 (1987) and Turk et al. Int. J. Artificial Organs 21:274-278 (1998)). To our knowledge however no studies have addressed the effect of zinc supplementation on mucosal immune responses to orally administered antigens in zinc-replete adults.

We have now found that enteric administration of zinc compounds favourably modulates the immunogenic effect of enterically administered antigens in healthy, i.e. zinc replete, adults.

Thus viewed from one aspect the invention provides the use of a physiologically tolerable zinc compound (e.g. a water-soluble zinc salt or complex) for the manufacture of a medicament for use in a method of treatment of an animal (preferably a mammal, more preferably a human) to generate a mucosal immune response therein to an antigen, which method comprises administering to said animal a said physiologically tolerable zinc compound and a said antigen whereby to generate said response, preferably by bringing said zinc compound and said antigen into contact with a mucosal surface in said animal.

Viewed from a further aspect the invention provides a method of treatment of an animal (preferably a mammal, more preferably a human) to generate a mucosal immune response therein to an antigen, which method comprises administering to said animal an effective amount of a physiologically tolerable zinc compound (e.g. a water-soluble zinc salt or complex) and an effective amount of a said antigen whereby to generate said response, preferably by bringing said zinc compound and said antigen into contact with a mucosal surface in said animal.

Viewed from a further aspect the invention provides an enterically administrable pharmaceutical composition comprising an antigen, a physiologically tolerable zinc compound, and optionally at least one physiologically tolerable carrier or excipient.

Viewed from a still further aspect the invention provides a kit comprising a first enterically administrable pharmaceutical composition comprising an antigen and optionally at least one physiologically tolerable carrier or excipient, and a second enterically administrable pharmaceutical composition comprising a physiologically tolerable zinc compound and optionally at least one physiologically tolerable carrier or excipient.

Viewed from a yet still further aspect the invention provides the use of an antigen (preferably a pathogen antigen or an allergic reaction inducing antigen) for the manufacture of a medicament for use in a method of treatment of an animal (preferably a mammal, more preferably a human) to generate a mucosal immune response therein to said antigen, which method comprises administering to said animal a physiologically tolerable zinc compound and a said antigen whereby to generate said response, preferably by bringing said zinc compound and said antigen into contact with a mucosal surface in said animal.

Viewed from another aspect the invention provides the use of a physiologically tolerable zinc compound (e.g. a water-soluble zinc salt or complex) for the manufacture of a medicament for use in a method of treatment of an animal (preferably a mammal, more preferably a human) to inhibit systemic immune system function therein, e.g. in conjunction with organ transplant or autoimmune or allergic disease treatment.

Although the direct contact of the zinc compound with the mucosal surface is a preferred feature, the zinc may exert its effect by other means, for example, systemically and locally.

As used herein, the term “animal” covers mammals, domestic animals, birds, fish, shellfish, humans etc.

In the methods of the invention, the zinc compound and antigen may be administered together or separately; preferably the zinc compound is administered one or more (e.g. 1, 2, 3 or 4) times daily, preferably after (e.g. one to twelve days, more preferably 9 days after) the first antigen administration, more preferably over a period commencing before and concluding after the or each administration of the antigen. The antigen is preferably administered at least twice, e.g. at intervals of 10 to 30 days. Particularly preferably zinc administration is over a period of several days (e.g. 1 to 7 days) before and after antigen administration. Desirably the zinc compound and the antigen are administered to the same type of mucosal surface, e.g. by oral, rectal, vaginal, nasal, sublingual administration or by administration into the lungs or trachea. Oral, rectal and vaginal administration, especially oral administration are particularly preferred.

The antigen and the zinc compound may, as indicated above, be administered together or separately. The administration form may be any one suitable for the intended administration route, e.g. tablets, coated tablets, capsules, solutions, suspensions, syrups, dispersions, sprays, powders, suppositories, pessaries, etc and conventional pharmaceutical carriers and excipients may be used. The zinc and/or antigen may moreover be formulated together with a foodstuff or foodstuff additive. Administration forms conventional for oral, rectal or vaginal administration of pharmaceuticals are preferred, e.g. tablets, capsules, solutions, suspensions, powders, suppositories and pessaries.

The antigen used according to the invention may be any antigen capable alone, or in conjunction with zinc treatment, of eliciting a mucosal immune response; it is particularly preferably an antigen associated with a pathogen which infects via the mucosal surfaces of the body, e.g. a virus, bacterium, yeast or fungus. The antigen in this case may be a live pathogen, or a compound (e.g. a coat protein or lipid) characteristic of the pathogen, or an inactivated, or a weakened or killed form of the pathogen. Examples of typical pathogens which may be vaccinated against using the method of the invention thus include bacteria (such as those responsible for cholera, typhoid fever, dysentery and E.Coli diarrhoea and Chlamydia urethritis or pelvic inflammatory disease), viruses (such as rhinoviruses, influenza viruses, polio viruses, rotavirus, and especially sexually transmitted viruses (e.g. hepatitis B, herpes viruses, human papilloma viruses, HIV, etc), fungi (such as Candida albicans) and protozoa (such as Entamoeba histolytica and Cryptosporidium parvum).

Alternatively, however, the method of the invention may be used to build up tolerance to a chemical rather than immunity to infection. Such chemicals may be ones either associated or not associated with pathogens, e.g. chemicals produced by pathogenic bacteria in the gut (e.g. toxins)or chemicals which produce an allergic response such as foreign proteins in household dust, pet hair or skin flakes, pollens, food, etc.

Surprisingly, zinc supplementation appears to reduce the immune response to certain antigens, notably the B-subunits of the cholera toxin, and in an alternative embodiment of the invention zinc may be given in conjunction with organ transplantation or other events where systemic immune response suppression is desired, optionally in conjunction with administration of an immune system suppressor such as cyclosporin and the other immunosuppressants listed in Martindale. The Complete Drug Reference, 32nd Edition, Pharmaceutical Press, London, 1999, for example. Indeed, use of zinc in this manner may have the beneficial effect of reducing the amount of cyclosporin required.

The dose of the antigen given according to the invention will depend on the particular antigen and its status (e.g. live or killed); however suitable dosages may readily be determined using conventional techniques, e.g. dose response curves for doses increasing from ineffective towards effective, extrapolation from animal models, etc.

The dosage of the zinc given according to the invention will generally be in the range 1 to 1000 mg elemental zinc per day, preferably 5 to 250 mg Zn/day especially 10 to 150 mg Zn/day. Once again suitable dosages may be determined by conventional techniques, e.g. as mentioned above.

The zinc compound administered according to the invention may be any physiologically tolerable (i.e. non-toxic) salt or complex from which zinc is bioavailable. Preferably the compound is water-soluble; however lipid soluble zinc complexes may be used. In one preferred embodiment, the zinc compound is formulated in a sustained release form, e.g. in liposomes, liquid crystals or multipellet-in-capsules forms. Typical salts and complexes include inorganic and organic salts such as zinc sulphate, zinc acetate, zinc acexamate, zinc amino acid chelates, zinc aspartate, zinc chloride, zinc gluconate, and zinc oxide.

The invention is particularly applicable both to patients with and without zinc deficiency, as may be reflected in serum zinc concentrations respectively below or above 10 μM, preferably above 12 μM.

One aspect of the present invention thus relates to synergistic immunological adjuvant formulations for potentiating a mucosal immune response. The formulations comprise an antigen delivered during a period of zinc supplementation.

Similarly, a second aspect of the present invention is related to a method of immunising a host where the said method comprises administering to said host:

a) an antigen capable of providing an immune response in said host; and

b) an immunomodulating formulation comprising zinc ions.

Likewise a further aspect of the present invention relates to a formulation and method that increases mucosal responses to an antigen and decreases systemic response to the antigen.

Another aspect of the present invention relates to a method of suppressing harmfully exaggerated immune responses to otherwise innocuous substances or to microorganisms, e.g. by the administration of a compound selected from the group comprising zinc sulphate, zinc gluconate, zinc oxide and zinc chloride and other non-toxic zinc-containing compounds.

The publications referred to herein are hereby incorporated by reference.

The invention will now be described further with reference to the following non-limiting Example and the accompanying drawings wherein:

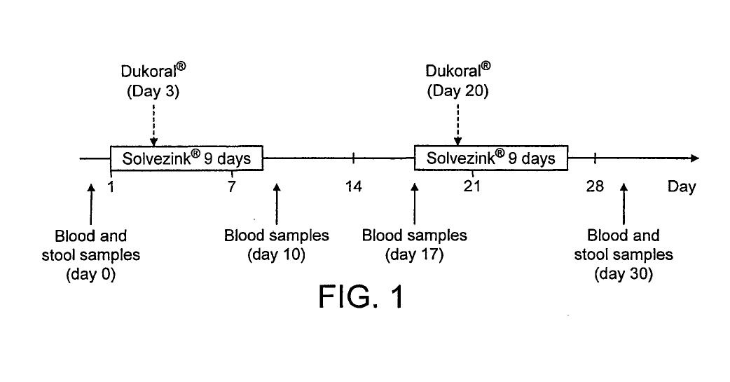

FIG. 1 shows zinc administration, immunisation and sampling schedule.

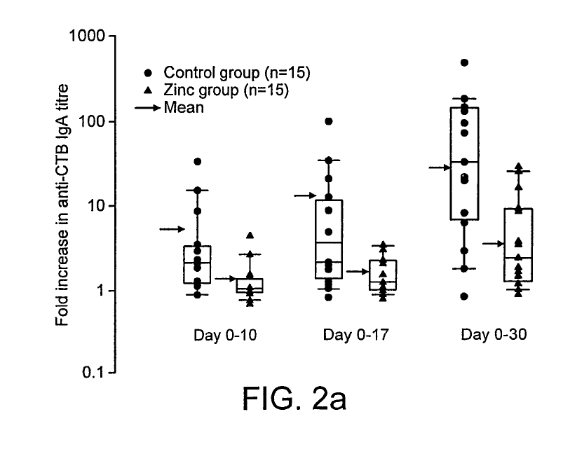

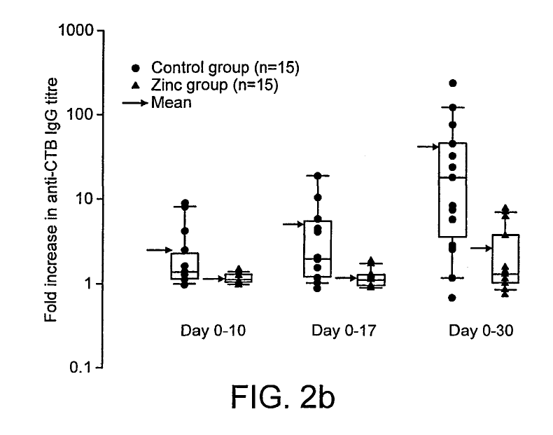

FIG. 2 shows fold serum anti-CTB IgA (a) and IgG (b) titer rises 7 (day 10) and 14 (day 17) days after one dose and 10 (day 30) days after a second dose of an oral killed CTB-whole-cell cholera vaccine in zinc supplemented (▾) and non-supplemented (●)vacinees

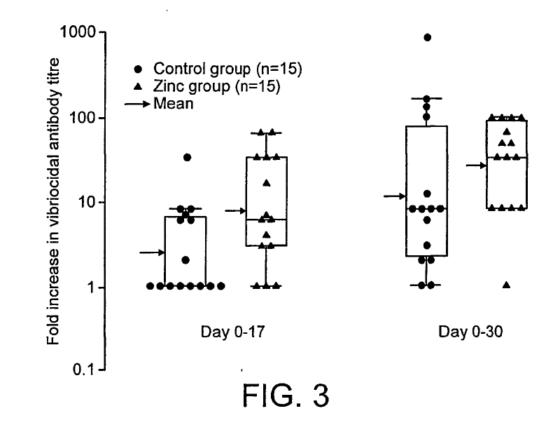

FIG. 3 shows the fold vibriocidal antibody titer rise 14 (day 17) days after the first and 10 (day 30) days after the second dose of an oral killed CTB-whole-cell cholera vaccine in zinc supplemented (A) and non-supplemented (●)vacinees.

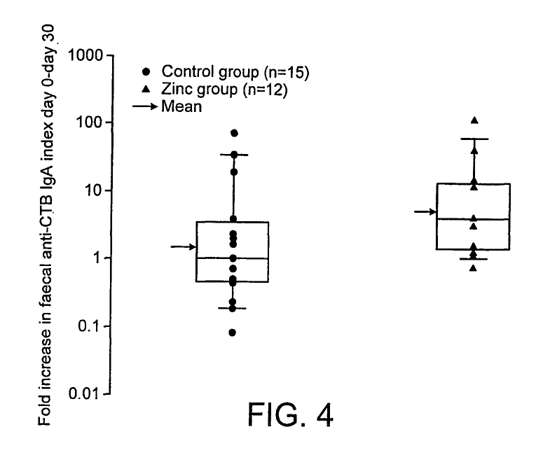

FIG. 4 shows fold faecal anti-CTB IgA index rise in zinc supplemented (▾) and non-supplemented (●)volunteers immunised with an oral killed CTB-whole-cell cholera vaccine.

### EXAMPLE

### Materials and Methods

Study Design

Thirty medical students aged (20 to 29 years) were enrolled in the study. None of the volunteers had an ongoing disease, nor were they immunised against cholera previously and none of them had travelled outside the Scandinavian countries in the 6 months prior to the study. The trial was performed in an open randomised manner.

Vaccine and Zinc Administration

The study participants were immunised orally with Dukoral® (SBL Vaccin AB, Stockholm, Sweden). A zinc sulphate preparation Solvezink® (Tika Läkemedel AB, Lund, Sweden) was used for zinc-supplementation, each effervescent tablet containing 200 mg of zinc sulphate, corresponding to 45 mg of elemental zinc. Each vaccine dose, containing 1 mg of recombinately produced cholera B subunit toxoid (CTB) and 10¹¹ killed O1 vibrios, was administered in 150ml of a sodium bicarbonate solution.

The participants were divided by block randomisation into a group that received zinc-supplementation and a control group. Both groups were given Dukoral® twice with a 17-day interval between the first immunisation and the booster dose. The immunisations were done in plenary sessions to ensure maximum compliance, at least 1 hour before or 2 hours after any meal and not in conjunction with zinc administration (to minimise interactions between the vaccine and food substances and pancreatic enzymes on the one hand and with zinc on the other).

The zinc-supplementation group received Solvezink® for two periods of 9 days (FIG. 1). In this period one effervescent tablet of Solvezink® was administered after a meal three times daily. Subjective side effects to both Dukoral® and Solvezink® were recorded during the periods of vaccine- and zinc-administration. Four months after the trial, any events of illness were retrospectively registered.

Sampling and Processing of Specimens

Faecal Samples

Stool samples were obtained from the participants on the day before the first immunisation (day 0), and 10 days after the booster immunisation (day 30)(FIG. 1). Approximately 2 g of freshly voided faeces was collected by the vaccinees at home in pre-weighed 20 ml plastic tubes (Nalge Nunc International, Naperville, USA) less than 12 hours prior to the collection of blood samples. To preserve antibodies in the faecal specimens, the vaccinees were instructed to immediately store the plastic tubes at the lowest available temperature (refrigerator temperature at +4° C. or in a home-freezer temperature at −20° C.). Upon delivery (within the next 12 hours) the samples were transferred to −70° C.

After recording the net weights for each stool sample, faecal extracts (FEs) were made (essentially as described by Grewal et al. in J. Immunol. Methods 239:53-62 (2000)) by adding 4.1 ml PBS (pH=7.2) containing proteolytic enzyme inhibitors 0.2 mM 4-(2-aminoethyl)-benzenesulphonylfluoride (Calbiochem-Novabiochem Corp., La-Jolla, USA), 1 μg/ml aprotinin (Sigma Chemical Co., St. Louis, USA), 10 μM leupeptin (Sigma Chemical Co.), and 3.25 μM bestatin (Boehringer Mannheim, Indianapolis, USA) per gram faeces. The solid matter was suspended by extensive vortexing followed by centrifugation at 13,500 rpm at −-20° C. for 15 minutes. The clear supernatants were subsequently stored in aliquots at −70° C.

Serum Samples

Serum for immunological and biochemical analyses was obtained by venous puncture on days 0, 17 and 30 and in addition, 1 day after the first period of zinc administration (day 10) (FIG. 1) and stored in aliquots at −70° C. To minimise effects of diurnal variation on serum zinc concentration, the serum samples were collected at the same time of the day.

Determination of Anti-CTB IgA and IgG in Serum

Anti-CTB IgA and IgG in serum were determined by a GM1-ELISA as described by Svennerholm et al. in J. Infect. Dis. 147:514-522 (1983). Anti-CTB titres in the samples were assigned absolute units (U) based on end-point titrations of a high-titred reference serum sample included in each plate to compensate for variations between analyses on different occasions. All samples from each vaccinee were analysed on the same plate.

Anti-CTB IgA and Total IgA Concentrations and Vibriocidal Antibodies in Faecal Extracts

Levels of serum antibodies against the bacterial whole cell components were determined using a vibriocidal assay (see McIntyre et al., J. Infect. Dis., 1: 468-475 (1964)). Samples were two-fold serially diluted from an intial dilution of 1:5 and endpoint titers determined as the reciprocal dilution of the test samples giving complete bacterial activity. Titers of <5 U/ml were assigned the value 2.5 U/ml)

Anti-CTB Iga in the FEs were determined by the GM1-ELISA essentially as described for serum in Svennerholm et al. (supra), except that the sample dilution in the first well was 1:2 and that the samples were incubated overnight at +4° C. before bound immunoglobulins were detected. As for the serum samples, anti-CTB antibody titres in the samples were assigned absolute units (U) based on end-point titrations of a high-titred reference FE included in each plate. The detection limit of the faecal anti-CTB IgA ELISA corresponds to an OD₄₉₀ in the well with the lowest dilution (i.e. 1:2) of 0.2 above background, below which all samples were assigned the log titre 0. When the OD₄₉₀ in wells with the lowest dilution is between 0.2 and 0.4 above background, the estimates have a greater analytical uncertainty than when OD values are above 0.4 over background. We accordingly generated ordered titres for samples with first well OD₄₉₀ values between 0.2 and 0.4 above background. Thus, samples with first well OD values between 0.2 and 0.35 above background were assigned 0.15 in log titre, while samples with first well OD₄₉₀ values above 0.35 and up to 0.4 were given a log titre of 0.3. These ordered estimates for samples with first well OD₄₉₀ values between 0.2 and 0.4 above background allowed ranked data analysis to be undertaken and, after assessing the distributions, also allowed for multiple parametric regression analysis.

Concentrations of total IgA in the FEs were determined using a modified microplate ELISA method as described by Grewal et al. (supra). All faecal data from vaccines with a >10-fold variation in total IgA in FEs between pre- and post-immunisation samples were excluded from further analyses. Furthermore, specimens with total IgA concentration <10 μg/ml were excluded from further analyses. The specific IgA antibody titres were divided by the total IgA concentration in the FEs, obtaining the anti-CTB IgA index, thereby adjusting for variations in the IgA content in faecal samples collected from different vaccinees on different days (see Grewal et al. (supra)).

Biochemical Parameters

Serum C-reactive protein (s-CRP) was measured by an immunoturbidimetric assay from Orion Diagnostica, Espoo, Finland. Serum zinc (s-Zn) was determined by inductively coupled plasma atomic emission spectrometry (ICP-AES) from Thermo Jarell, MA, USA (Ash IRIS/AP), at a wavelength of 206.2 nm.

Statistical Methods

The Mann-Whitney U test was used for comparing the titre rises in the zinc and control groups for all immunological parameters. Independent t-tests were used to compare differences in s-Zn and CRP between the zinc and control groups.

Because of the ordered titres given to samples with first well OD values between 0.2 and 0.4 above background, the Spearman's rank correlation coefficient, rho, was computed to examine the association between anti-CTB responses (from day 0-30) in serum and in faeces. The distributions of the log transformed differences in anti-CTB titres in serum and in faecal anti-CTB indices were symmetrical. Accordingly, multiple linear regression of log serum anti-CTB responses on zinc and differences in log transformed faecal anti-CTB indices were used to examine the relationship between serum responses on the one hand and zinc supplementation and faecal responses on the other.

### Results

Pre-immunisation Status

Based on the predefined criteria described above, 5 zinc-supplemented vaccinees were excluded from the analyses of faecal immune responses. Thus, 4 had pre- and post-immunisation total IgA concentrations <10 4μg/ml while 1 had a pre-immunisation concentration >10 μg/ml and a post-immunisation concentration <10 μg/ml.

Prior to immunisation there were no significant differences in serum anti-CTB IgA or IgG titres or faecal anti-CTB IgA indices between zinc and control groups (Table 1). All vaccinees had s-Zn values within the normal range prior to intervention and there was no substantial difference in s-Zn between the groups (Table 2). Table 1: Pre-immunisation serum antitoxin titres, serum vibriocidal titers and faecal anti-CTB IgA indices in zinc supplemented and non-supplemented volunteers immunised with Dukoral®.

Pre-immunisation log titres

| | Zinc-group | Control-group |
|---|---|---|
| Immunological | Median log titre | Median log titre |
| parameter | (interquartile range) | (interquartile range) |
| Serum anti-CTB IgA log | 0.49(0.24-1.00) | 0.59(0.32-1.10) |
| titre | | |
| Serum anti-CTB IgG log | 0.75(0.45-0.89) | 0.83(0.67-0.97) |
| titre | | |
| Serum vibriocidal | 0.40 (0.40-1.6) | 0.40 (0.40-0.70) |
| antibody titer | | |
| Prpoortion with pre- | 4/15 | 1/15 |
| immunisation titer ≧40 | | |
| Faecal anti-CTB IgA | −0.97(−1.30-−0.50) | −0.75(−1.0-−0.092) |
| log index | | |

Table 2: Changes in serum zinc (s-Zn) and C-reactive protein in zinc-supplemented and in non-supplemented volunteers immunised with Dukoral®.

| | Zinc-group | | Control-group | |
|---|---|---|---|---|
| | | CRP | | CRP |
| | s-Zn | Normal | s-Zn | Normal |
| | Normal range | range <7 | Normal range | range <7 |
| | 10.0-16.0 μM | mg/l | 10.0-16.0 μM | mg/l |
| | Mean (95% CI) | Mean | Mean (95% CI) | Mean |
| Day | 11.3(10.3-12.2) μM | <7 mg/l | 12.4(11.5-13.4) μM | <7 mg/l |
| 0 | | | | |
| Day | 12.5(11.3-13.8) μM | <7 mg/l | 11.1(10.1-12.1) μM | <7 mg/l |
| 10 | | | | |
| Day | 11.5(10.5-12.5) μM | 17 mg/l | 11.4(10.2-12.5) μM | 10 mg/l |
| 17 | | | | |

Antitoxin Responses in Serum

The magnitudes of the serum anti-CTB IgA and IgG titre rises from day 0 to day 30 were significantly lower (p=0.004 and p=0.001, respectively) in the zinc supplemented group (median 2.3-fold and 1.3-fold, respectively) than in the control group (median 30.9-fold and 17.4-fold, respectively)(FIG. 2a and FIG. 2b). In the control group, the proportion of vaccinees with >2-fold increases in anti-CTB IgA and IgG titres was 87%. The rise in vibrocidal titers from day 0 to day 17 and day 0 to day 30 (FIG. 3) was higher in the zinc group (median 6-fold and 32-fold respectively) than in the control group (median 1-fold and 8-fold respectively).

Antitoxin Responses in Faecal Extracts

The rise in faecal anti-CTB IgA indices from day 0 to day 30 (FIG. 4) was higher in the zinc supplemented group (median 6.8-fold) than in the control group (median 1.6-fold), p=0.06. Forty-seven percent of the vaccinees in the control group showed a >2-fold increase in the anti-CTB IgA index.

Changes in Biochemical Parameters

S-Zn was within the normal range (10-17 μM) for all vaccinees on all sampling occasions. On day 10, the s-Zn levels were higher in the zinc supplemented group than in the control group (mean 12.5 μM vs. 11.1, p=0.06); this difference was not present on day 17 (Table 2). Changes in CRP after immunisation with Dukoral® were observed in both groups, however, there were no significant differences in CRP between the zinc- and the control group at any point of time (Table 2).

Reported Side Effects from Immunisation and Zinc Administration

Four out of 30 vaccinees found the taste of the Dukoral® solution unpleasant and 1 out of 30 experienced mild upper gastrointestinal disturbance following vaccine intake. Two out of 15 did not like the taste of the Solvezink® tablet. No serious illness or hospitalisations were reported during the 4-month follow-up period.

Association between Zinc Supplementation, Faecal Antibody Response and Serum Antibody Response

There was a negative correlation between anti-CTB response in faeces (day 0-30) and in serum (day 0-30) (Spearman's rho=−0.38, p=0.06). Because of the symmetric distribution of the log-transformed faecal and serum anti-CTB IgA responses, a univariate linear regression was used which yielded a regression coefficient of −0.37 (95% CI: −0.76, 0.2; p=0.06). Stratification on whether the subjects had been given zinc showed no effect measure modification between zinc supplementation and the association between faecal and serum anti-CTB responses. A bivariate linear regression with log anti-CTB serum response as the dependent and log faecal anti-CTB responses as well as zinc supplementation as independent variables yielded an R² of 29% (p=0.02). The regression coefficient (β) for faecal responses was −0.24(95% CI −0.62, 0.17) and for zinc supplementation −0.68(95% CI −1.35, −0.14).

The median serum anti-CTB IgA and anti-CTB IgG titre rises were both 13 times lower while the median intestinal anti-CTB IgA index rise was 4 times higher in the zinc group than in the group not receiving zinc. Student's independent-samples t-test, on data from the 25 subjects not excluded because of indeterminable faecal responses, showed that the serum geometric mean anti-CTB IgA titre rise was 7 (956 CI 1.6, 28) times lower while the intestinal geometric mean anti-CTB IgA titre rise was 4(95% CI 0.9, 19) times higher in the zinc group than in the group not receiving zinc.

The anti-CTB responses in faeces and in serum were negatively correlated (Spearman's rho=−0.38, β=−0.37 (95% CI: −0.76, 0.2; p=0.06)). Thus, in the study population, where 10 of 25 individuals received moderately high doses of zinc, a stronger intestinal immune response was associated with a poorer response in serum. After verifying that zinc supplementation was not an effect measure modifier on the association between faecal and serum responses, a bivariate regression analysis was used to evaluate the combined and adjusted effects of intestinal response and zinc supplementation on serum response. Zinc supplementation and intestinal response could explain 29% of the variation in serum responses [p=0.02]. Adjusting for zinc supplementation, intestinal response was non-significantly negatively associated with serum response [β=−0.24 (95% CI −0.62, 0.17, p=0.25], while adjusting for faecal response, zinc supplementation had a negative impact on serum anti-CTB responses [β=−0.68 (95% CI −1.35, −0.14, p=0.046)]. Thus, it seems that the zinc supplementation had a negative effect on the serum response to CTB and that only a part of this effect may have been mediated by an enhanced intestinal immune response.

Mucosal tolerance has hampered the development of effective mucosal vaccines as proteins applied to mucosal surfaces without specific immunomodulating substances are likely to induce unresponsiveness rather than immunity. Still, an important part of any mucosal immune response, whether tolerogenic or immunogenic in systemic terms, is the specific immunoexcluding barrier properties of local S-IgA production. The stronger faecal anti-CTB IgA responses in the zinc-group may reflect a tolerogenic process in part mediated by a strengthened local immune response in the intestinal mucosa. However, the analysis did not indicate that this was the main mechanism. The significant decrease in serum antitoxin responses in the zinc group can be explained by observations on in vitro lymphocyte culture studies where the monocyte-dependent T-cell stimulation and activation by zinc depends on the zinc concentration in a diphasic manner. Thus, by increasing the physiological concentration of zinc slightly, an increased T-cell responsiveness and activation can be observed. By further raising the zinc concentration in the monocyte/lymphocyte environment an actual inhibition of certain immune responses may occur.

Subjective side effects from both Solvezink® and Dukoral® were negligible in both groups. Neither group had an increased morbidity in the 4-month period following immunisation and zinc administration. Although minor changes in biochemical parameters were detected during the period of zinc administration and after vaccination, the values were within the normal range for all vaccinees on all sampling occasions. Immunomodulating substances suitable for clinical use are important for both immunogenic and tolerogenic mucosal vaccines currently under development and represent a large variety of compounds acting via many different pathways, however, all depending upon the selective activation of specific T-cell subpopulations controlling the immune responses. A major difficulty encountered during the search for immunomodulating substances is to achieve acceptable safety for clinical use. This trial supports the conclusion that the present dosage and duration of zinc administration seems to be well-tolerated in healthy adults.

In the group that did not receive zinc, the proportion of vaccinees that showed a >2-fold increases in anti-CTB IgA and IgG titres and a >4-fold increase in vibriocidal serum responses were in line with previous observations. On the other hand, the proportion of vaccinees with a >2-fold increase in intestinal anti-CTB index (47%) was somewhat lower than that reported previously. The termination of faecal antibody titres is subject to a higher degree of methodological error than that of serum titres. This lower precision is likely to be unrelated to zinc supplementation and intestinal or serum immune responses, as illustrated by the fact that 3 of the 5 excluded subjects had too low total IgA levels before the study was started. Therefore, the estimate of the intestinal anti-CTB IgA response is likely to have a somewhat lower precision that the estimate of serum IgA and IgG responses. The somewhat imprecise assessment of intestinal IgA responses may also have influenced the precision of the negative correlation between the intestinal and serum immune responses. Thus, the finding that the overall negative correlation between intestinal and serum immune responses was only to a small extent mediated by zinc supplementation my have been underestimated.

1. The use of a physiologically tolerable zinc compound for the manufacture of a medicament for use in a method of treatment of an animal to generate a mucosal immune response therein to an antigen, which method comprises administering to said animal a said physiologically tolerable zinc compound and a said antigen whereby to generate said response, preferably by bringing said zinc compound and said antigen into contact with a mucosal surface in said animal.

2. Use as claimed in claim 1 of a zinc compound selected from zinc sulphate, zinc gluconate, zinc oxide and zinc chloride.

3. Use as claimed in either of claims 1 and 2 wherein in said method the zinc compound is administered in a dose of 5 to 250 mg Zn/day.

4. Use as claimed in any one of claims 1 to 3 for the manufacture of an orally administered medicament.

5. Use as claimed in any one of claims 1 to 4 wherein in said method said antigen is or is a part of a pathogen.

6. Use as claimed in claim 5 wherein said antigen is or is a part of bacterial pathogen.

7. Use as claimed in claim 6 wherein said antigen is an orally administered cholera vaccine.

8. A method of treatment of an animal to generate a mucosal immune response therein to an antigen, which method comprises administering to said animal an effective amount of a physiologically tolerable zinc compound and an effective amount of a said antigen whereby to generate said response, preferably by bringing said zinc compound and said antigen into contact with a mucosal surface in said animal.

9. A method as claimed in claim 8 wherein said compound and said antigen are administered to said animal orally.

10. A method as claimed in claim 8 or claim 9 wherein said antigen is or is part of a pathogen.

11. An enterically administrable pharmaceutical composition comprising an antigen, a physiologically tolerable zinc compound, and optionally at least one physiologically tolerable carrier or excipient.

12. A kit comprising a first enterically administrable pharmaceutical composition comprising an antigen and optionally at least one physiologically tolerable carrier or excipient, and a second enterically administrable pharmaceutical composition comprising a physiologically tolerable zinc compound and optionally at least one physiologically tolerable carrier or excipient.

13. The use of an antigen for the manufacture of a medicament for use in a method of treatment of an animal to generate a mucosal immune response therein to said antigen, which method comprises administering to said animal a physiologically tolerable zinc compound and a said antigen whereby to generate said response, preferably by bringing said zinc compound and said antigen into contact with a mucosal surface in said animal.

14. The use of a physiologically tolerable zinc compound for the manufacture of a medicament for use in a method of treatment of an animal to inhibit systemic immune system function therein.
